(19) United States
(12) Patent Application Publication
(10) Pub. No.: US 2009/0000630 A1
(43) Pub. Date: Jan. 1, 2009

(54) Instant Face-Lifter

(76) Inventor: Ricardo Alfredo Fuenmayor Aray, San Carlos (VE)

Correspondence Address: Ricardo Alfredo Fuenmayor Aray, 2nd Floor, 6318 23 Ave., Brooklyn, NY, 11204, US

(21) Appl. No.: 11/986,714

(22) Filed: Nov. 26, 2007

Related U.S. Application Data
(60) Provisional application No. 60/905,057, filed on Mar. 5, 2007.
(60) Provisional application No. 60/904,996, filed on Mar. 5, 2007.
(60) Provisional application No. 60/995,258, filed on Sep. 25, 2007.

(51) Int. Cl. A61B 19/00; A61F 2/02

(52) U.S. Cl. 128/899; 623/11.11

(57) ABSTRACT

Following are six improvements on the prior art entitled “Instant Face-Lifter”. “Intra-scalp TV/Radio/Stereo-Music-Reproducing Device Face-Lifter”: uses an intra-scalp silicone Face-Lifter as stand for an implanted TV set and/or AM-FM Radio and/or an Open Platform Stereo Music device (MP3, MP4 Ipod®, Walkman®, WMA®). “Intra-scalp GPS-Face-Lifter”: a Face-Lifter as a stand for a GPS device. “Intra-scalp Heart Pace-Maker-Face-Lifter”: a Face-Lifter as a stand for a heart pace-maker device. “Intra-scalp Artificial Hair Anchoring Base Face-Lifter”: a Face-Lifter as an anchoring base for permanently implanting small bundles of artificial hair on balding people. “Intra-scalp Bullet-Proof Head-Armor Face-Lifter”: a Face-Lifter as bullet-proof head armor. “Intra-scalp Orgasm-inducer Face-Lifter”: a Face-Lifter as a stand for an orgasm-inducing electric device.

Field of Use No. 1: “Intra-scalp TV/Radio/Stereo-Music-Reproducing Device Face-Lifter”: Using a basic Face-Lifter as a stand or support for an implanted TV signal receptor set and/or an AM-FM Radio Signal Receptor set and/or an Open Platform Stereo Music Producing device (i.e. MP3, MP4, Ipod®, Walkman®, WMA®, etc).
Field of Use No. 2: “Intra-scalp GPS-Face-Lifter”: Using a Face-Lifter as a stand or support for a miniature GPS device for satellital follow-up of at-risk personnel and as anti-kidnapping device.
Field of Use No. 3: “Intra-scalp Heart Pace-Maker-Face-Lifter”: Using a Face-Lifter as a stand or support for a heart pace-maker device.
Field of Use No. 4: “Intra-scalp Artificial Hair Anchoring Base Face-Lifter”: Using a Face-Lifter as an anchoring base for permanently implanting small bundles of artificial hair on balding people.
Field of Use No. 5: “Intra-scalp Bullet-Proof Head-Armor Face-Lifter”: Using a Face-Lifter as bullet-proof head armor against gunshot attacks to the head.
Field of Use No. 6: “Intra-scalp Orgasm-inducer Face-Lifter”: Using a Face-Lifter as a stand or support for an orgasm-inducing electric device for anorgasmic women.
(Note 1: An “Instant Face-Lifter” a beautifying device, consists of an intra-scalp oval-shaped device made of solid, medical grade silicone, typically measuring 5 12/16″ long×4 12/16″ wide, with sloping edges and an ever increasing thickness in the direction from border to center starting at a negligible thickness at the borders and increasing to a varying maximum thickness—i.e. ½″, ¾″, 1″, 1½″, etc—at its center as to be able to provide the user the desired amount of skin stretching effect”)
(Note 2: The “Instant Face-Lifter”—in this case the prior art—, has been filed previously, under the Provisional Application No. 60/905,057, Filing Date Mar. 5, 2007, Confirmation No. 2853, Name of Inventor: Ricardo A. Fuenmayor A., Title of Invention: “Instant Face-lifter”. Later, I re-applied as a Non-Provisional Application for this invention (Ser. No. 11,993,291, on Aug. 15, 2007)).
(Note 3: There is also a prior Provisional Application No. 60/904,996 for three other improvements on the Face-Lifter, Filing Date Mar. 5, 2007, Confirmation No. 3578, Name of Inventor: Ricardo A. Fuenmayor A., Title of Invention: Instant Face-lifter. Furthermore, there is currently a Non-Provisional Application being requested for these three prior improvements (Ser. No. 11,893,310, on Aug. 15, 2007)).
(Note 4: Additionally, a Provisional Application No. 60/995,258 for six other improvements on the Face-Lifter has been filed: Filing Date Sep. 25, 2007, Confirmation No. 2352, Name of Inventor: Ricardo A. Fuenmayor A., Title of Invention: Instant Face-lifter.)

### BACKGROUND OF THE IMPROVEMENTS OF THE INVENTION

The field of endeavor to which the invention pertains, based on the U.S. patent Classification Definitions, is Class 128, Surgery, subclasses for apparatus which assists a body part or function but does not physically replace or partially replace any normally existing body part, and Sub-Class 899, devices placed entirely within body and means used therewith. This subclass is indented under subclass 897. Subject matter including devices not elsewhere classifiable which are placed entirely within the body either through insertion through natural body openings or by surgical implantation and means used therewith.

Since there are several “Fields of Use” or different applications for the six (6) proposed improvements, each Field of Use's Background will be described separately in the following pages.

### Field of Use No. 1

In modern age, it is ever more prevalent the miniaturization of all types of electronic devices. Coupled to this fact, we, as a consumer society, have seen the onrise of all types of high fidelity music and sound reproducing equipments. Additionally, one cannot be a well-informed person without being heavily influenced by what is broadcasted on the different TV stations all over the world. Because of its unique characteristics (its strategic location, its conveniently concealed position, its appropriate height—up to one inch and a half thickness-, and its closeness to the main sensorial organs of sight and hearing) make the Face-Lifter a very versatile launching pad for multiple applications. In the Field of Use No. 1, the improvement on the prior art consists of using a basic Face-Lifter as a stand or support for an implanted TV signal receptor set and/or an AM-FM Radio Signal Receptor set and/or an Open Platform Stereo Music Producing device (i.e. MP3, MP4, Ipod®, Walkman®, WMA®, etc).

The improvement consists of a basic intra-scalp Face-Lifter, in whose silicone matrix has been embedded: a) a TV signal receiving set, and/or an AM/FM radio signal receptor, and/or an open platform stereo music reproducing device (i.e. MP3, MP4, Apple's® Ipod®, Microsoft's® WMA®, Sony's® Walkman®, etc.) with Bluetooth®-like or similar transmitter; b) a rechargeable battery (its energy provided by an electricity-producing motion-device similar to Seiko's® “Kinetic”® watches), c) two lateral sub-dermal 4″ long×½″ wide silicone side straps, each carrying within an earphone, on-line flat membrane-type switches (for On-Off function, volume control, silent switch, select mode switch) and wiring that continue their downward descent until reaching both ear canals, and/or d) Bluetooth®-like wireless earpieces (to do away with the use of straps).

Additionally, the improvement presents a handy remote control whereby one can select channels, choose mode of operation, set volume, turn it on-off, etc.

The TV receptor embedded in the face-lifter may transmit TV images wirelessly (i.e. via Bluetooth® transmitters and earpieces, and/or microwave, infrared or ultrasound waves) to a remote few inches wide flat screen mounted on a glasses frame in front of the eyes.

Among its many advantages, this improvement allows the user to listen hand-freely to his/her favorite music at will, as well as to keep him/her informed at all times, in an unnoticeable fashion, with an all-weather and water-proof technology that may be up-graded with other perks such as “wake-up call” features and others.

As the prior art, the improvement consists of a basic intra-scalp Face-Lifter, an oval-shaped device made of solid medical grade silicone, typically measuring 5 12/16″ long×4 12/16″ wide, with sloping edges and an ever increasing thickness in the direction from border to center, starting at a negligible thickness at its borders and increasing to a maximum thickness of 1½″ at the center, in whose silicone matrix has been embedded a TV signal receiving set, and/or an AM/FM radio signal receptor, and/or an open platform stereo music reproducing device (i.e. MP3, MP4, Apple's® Ipod®, Microsoft's® WMA®, Sony's® Walkman®. The invention is also sided under the scalp, in the space between the skin of the scalp and the galea aponeurotica of the user through a 3″ to 4″ incision made on the parietal area using local anesthesia. Furthermore, the device, once in place, still fulfills its beautifying purpose for people aged between 45-60 years old allowing for a skin-stretching action, whereby reducing forehead wrinkles and providing an instant fresher look.

### Field of Use No. 2

These are trying times. Due to many different reasons, and specially abroad, key people and personnel live under a constant pending menace of being forcefully “disappeared” by means of kidnappings, terrorist acts, soldiers being left behind enemy lines, or simply by being in the wrong place at the wrong time (i.e. tourists and/or journalists visiting dangerous countries or in turmoil). In the Field of Use No. 2, the improvement on the prior art consists of using a basic Face-Lifter as a stand or support for an implanted miniature GPS (“Global Positioning System”) device.

The improvement consists of a basic intra-scalp Face-Lifter, in whose silicone matrix has been embedded a GPS device and a rechargeable battery (its energy provided by an electricity-producing motion-device similar to Seiko's® “Kinetic”® watches).

Among its many advantages, this improvement would allow for a completely concealed and discreet way to follow-up all American personnel working or traveling abroad, specially those visiting dangerous places of the world (Iraq, Colombia, etc.).

Among its potential users:
Diplomatic personnel,
tourists traveling to dangerous places of the world,
US Army personnel (thus becoming a N.G.L.B. “Nobody Gets Left Behind” device),
firemen,
armour-car personnel, and
mountain climbers.

The intra-scalp GPS-Face-Lifter also works well as Anti-kidnappings device, allowing to abort kidnappings by serving as tracking device and allowing for the precise satellital location of missing persons, while keeping its beautifying properties to users in their fourth to fifth decade by gently stretching their face skin, further reducing eyebrow wrinkles and providing an instant fresher look.

### Field of Use No. “3”

The first cause of death among people of the Western world is directly related to heart or cardiovascular system failure. One of the ways modern science has found to counteract failing electric signals from the brain to the heart muscle that lead to or erratic heart beats is the use of implanted pace-makers: small electrical devices carrying leading wires generally implanted sub-dermally in the chest area as to assure the maintenance of the heart's appropriate rhythmic pace. In the Field of Use No. “3”, the Improvement on the prior art consists of using a intra-scalp Face-Lifter as a stand or support for an Implanted Heart Pace-Maker.

The improvement consists of a basic intra-scalp Face-Lifter, in whose silicone matrix has been embedded a Heart Pace-Maker, a rechargeable battery (its energy provided by an electricity-producing motion-device similar to Seiko's® “Kinetic”® watches) and sub-dermal leading wires that end up in the heart muscle.

This improvement's novelty consists of its innovative location which brings several important advantages: Because of its concealed location, the pace-maker Face-Lifter goes totally unnoticed, presenting no bulging on the chest area as the conventional model does, thus raising the user's self-esteem. Additionally, the improvement, being located on the top of the head, takes advantage of the continuous rotational head motion to generate energy by means of an ingenious rotating weigh combined with a tiny gearbox that converts few r.p.m. of the weigh's gyration movement into thousands of r.p.m. that impulses a small electricity-generator (a motion-device similar to Seiko's® “Kinetic”® watches). Importantly, the device, possessing rechargeable batteries, eliminates the need to have periodic surgeries to have the device removed at intervals to reinstall fresh batteries.

### Field of Use No. 4

It is a hard fact to accept to a significant percentage of the male population that due a variety of reasons (i.e. genetic heritage, hormonal reasons, old age and/or skin diseases), as they mature into old age, many of them will start to lose considerable amounts of hair from the top of their heads to different degrees, ranging from the development of clear spots until a totally bald condition. The improvement on the prior art consists of using a basic intra-scalp Face-Lifter as a Anchoring Base for Implanting Small Bundles of Artificial Hair as to allow the balding user to recover, in a permanent fashion, an as abundant amount of hair as he wishes to.

The improvement consists of using the basic intra-scalp Face-Lifter device as an Artificial Hair Anchoring Base, i.e. using a Face-Lifter as an anchoring base for permanently implanting small bundles of artificial hair. Each little bundle of artificial hair, containing between 3-12 strands of hair, ends up in a little inverted and sharp plastic hook, very much like a straightened fishing hook. The inverted sharp hook's goal is to penetrate through the scalp and into the structure of the Face-Lifter, anchoring itself to the silicon matrix, thus rendering it as a permanent fixture.

Using this improvement as an Artificial Hair Anchoring Base Face-Lifter will improve the Face-Lifter's basic beautifying properties of stretching the skin of the user's face, already in his forties, thus smoothening out expression lines and softening crow's feet, by additionally helping raise the user's self-esteem by rejuvenating him, allowing him to recover a furred or hairy look, as it was in his early youth.

### Field of Use No. 5

An alarming number of head wounds and injuries are produced by projectiles, i.e. gunshots, in USA, every year. Taking advantage of the fact that the skin covering most of the head is loosely bound to the skull bone, our aim is to slip below the scalp a sizeable piece of bullet-proof material which has been embedded into the structure of a Face-Lifter. In the Field of Use No. 5, the improvement on the prior art consists of using a Face-Lifter as a Bullet-Proof Head Armor Device.

The improvement consists of a thinner and larger version of the under-the-scalp Face-Lifter for covering a larger amount of head surface, as to be able to minimize the possible damages inflicted by a gunshot attack on the head. In its bullet-proof head armor application, a sizeable piece of bullet-proof Kevlar® and/or Zylon® fabric, sandwiched within a Face-Lifter, is implanted under the scalp as to surround most of the gray-matter areas of the brain, thus covering, and therefore protecting, the temporal, occipital, parietal, and frontal areas of the head against gunshot attacks.

Typical users of this improvement would be high-risk Army personnel, as well as Police officers, body guards, key politicians, diplomatic personnel assigned to dangerous places abroad, and anyone who feels that his/her physical integrity is being seriously threatened of suffering bodily harm.

Among its advantages are its concealment, being totally undetected from the outside, its shielding ability, thus diminishing the chances of projectiles entering into the head, and its great capacity to minimize the loss of gray matter should a gunshot attack be inflicted upon the user.

### Field of Use No. 6

There is an undetermined percentage of anorgasmic women in the US today (women that, due to physiological or psychological reasons, are incapable of reaching climax during sexual intercourse), with statistics of their real prevalence among the female population being unclear due to a lack of an appropriate psychiatric and psychological follow up. Being orgasms a matter of physiological stimulation of the proper nerve endings, it is possible for anorgasmic women to voluntarily self-induce climaxes by the electrical stimulation of the appropriate nerves that branch out from the lumbar section of the vertebral column and go directly to the vaginal and clitoral area of the body. In the Field of Use No. 6, the improvement on the prior art consists of using a Face-Lifter as a stand or support for an orgasm-inducing electric device.

The improvement consists of a basic intra-scalp Face-Lifter, in whose silicone matrix has been embedded a small-pulse electric generating device, (being in fact, simply a small rechargeable battery, its energy provided by a head-motion electricity-producing motion-device similar to Seiko's® “Kinetic”® watches), a sub-dermal membrane-type flat switch, and a sub-dermal 20-30 inch-long wiring that continue its downward descent until reaching the lumbar area of the back until getting in contact with the appropriate nerves leaving the vertebral column in order to attain the appropriate vaginal and clitoral nerve ending stimulation.

The advantages of using a Face-Lifter as a Intra-scalp Orgasm-inducer Face-Lifter are only obvious to anorgasmic women who have spent their entire life having children and having intercourses with their jolly husbands without understanding what is all the fuss about, becoming full of apathy toward their sexual partners and losing an important part of the joy of life due to their lack of an adequate physiological response. Once these women, by simply pressing a sub-dermal membrane-type flat switch on their cheeks at the appropriate moment of the intercourse, receive the benefits of reaching repeated climaxes, their sexual and marital lives will simply change forever for good. They will be able to voluntarily induce orgasms on themselves by simply pressing a button! Consequently, society at large will benefit, saving from potential breakage a number of marriages due to sexual insatisfaction, and ending up with happier husbands and more fulfilled housewives.

The advantages of this improvement are its concealment, since the device cannot be detected from the outside, having no noticeable wirings that would give out its presence, therefore raising the user's self-esteem and confidence; additionally, it gives the women users the ability to control their orgasms, thus becoming self-sufficient and independent of the love-making skill of their sexual partner; and, by using the orgasm-inducing electric device she also gets the benefits of using the Face-Lifter, in whose silicone structure the electric pulse generator is embedded, among these advantages: she'd get an instant face-lifting since the device dramatically improves a 40 to 50-year-old face by creating a brighter, softer, fresher appearance and restoring a more youthful, rested, and aesthetic look, by elevating drooping eyebrows; softening of forehead frown lines, and smoothening out vertical lines between the eyebrows, with minimum scarring or possibility of deforming hairline.

### BRIEF SUMMARY OF THE IMPROVEMENT ON INVENTION

Field of Use No. 1 uses a basic intra-scalp silicone Face-Lifter as a stand for a TV or AM-FM Radio signal receptor set and/or an Open Platform Stereo Music Producing device (i.e. MP3, MP4, Ipod®, WMA®, etc) with Bluetooth®-like transmitter and earpieces (and/or sub-dermal earphones), as to be able to listening hand-freely to recorded music and/or radio station and/or be able to watch TV or video images transmitted wirelessly to small screens held in front of the user's eyes by means of a glasses frame.

Field of Use No. 2 uses a basic intra-scalp silicone Face-Lifter as a stand for a miniature GPS device that works as an anti-kidnapping device and to help locate soldiers and the like who i.e. may have gotten lost behind enemy lines.

Field of Use No. 3 uses a basic intra-scalp silicone Face-Lifter as a stand for a rechargeable-battery heart pace-maker device endowed with a motion electricity-producing device, thus guaranteeing a long-lasting service, in a concealed place, without the need to regularly servicing it to put fresh batteries.

Field of Use No. 4 uses a basic Face-Lifter as an anchoring base for fastening small bundles of artificial hair provided with little hooks at their base, inserting them through the scalp and into the silicone matrix of the Face-Lifter structure as to allow balding users to enjoy a permanent hair transplant.

Field of Use No. 5 consists of a thinner and larger version of the Face-Lifter, thus covering a larger amount of head surface, its silicone matrix sandwiching a piece of bullet-proof Kevlar® and/or Zylon® fabric, implanted under the scalp in such a way as to surround most of the gray-matter areas of the brain, thus covering, and therefore protecting, the temporal, occipital, parietal, and frontal areas of the brain against gunshot attacks.

Field of Use No. 6 uses a basic intra-scalp silicone Face-Lifter as a stand for a small-pulse electric generator (basically, a small rechargeable battery with an electricity-producing device) whose purpose is to voluntarily self-induce climaxes in anorgasmic women with the help of a sub-dermal switch to electrically stimulate (by means of electrodes) the appropriate nerves that branch out from the lumbar section of the vertebral column and lead to the vaginal and clitoral area of the body when they are engaged in the sexual act with their husbands.

### BRIEF DESCRIPTION OF THE SEVERAL VIEWS OF THE DRAWINGS

Listing of figures: One Sheet of Drawings for each Field of Use. Six Fields of Use: Sheets of Drawings 1 thru 6.

Field of Use No. 1: Sheet 1/6. 2 Figures.

FIG. 1. Section View

It corresponds to a sectional cut through the middle of the device along its longest axis. It consists of a basic intra-scalp silicone Face-Lifter as a stand for a TV or AM-FM Radio signal receptor set and/or an Open Platform Stereo Music Producing device (i.e. Mp3, Mp4, Ipod®, WMA, etc) with Bluetooth-like transmitter and earpieces (and/or sub-dermal earphones), to listen to either recorded music and/or radio station and/or be able to watch TV programs on small screens held in front of the user's eyes by means of a glasses frame, hands free.

FIG. 2. Elevation View

This drawing corresponds to the depiction of the invention as seen from above.

Field of Use No. 2: Sheet 2/6. 2 Figures.

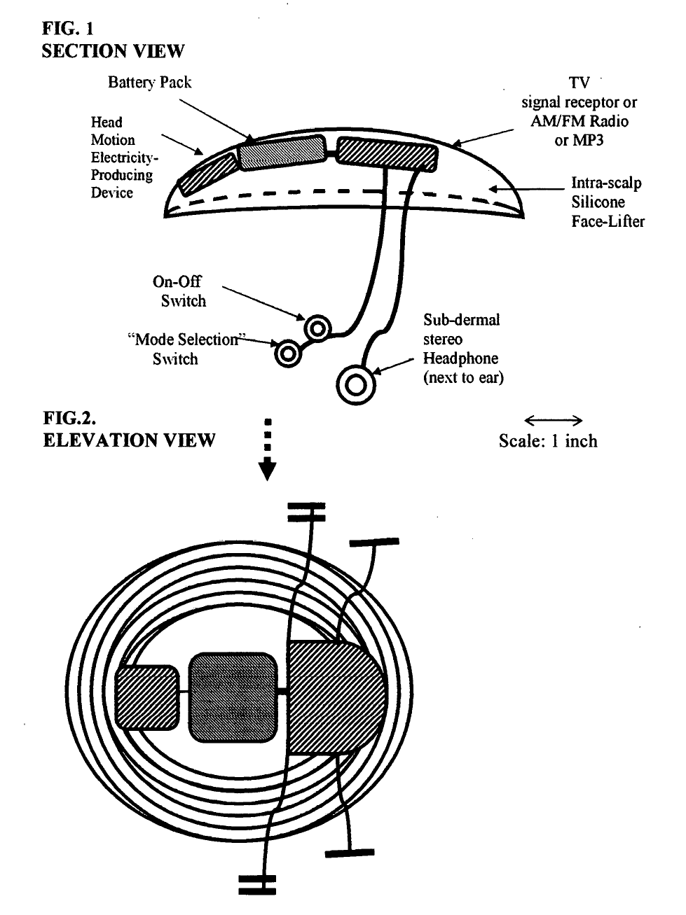

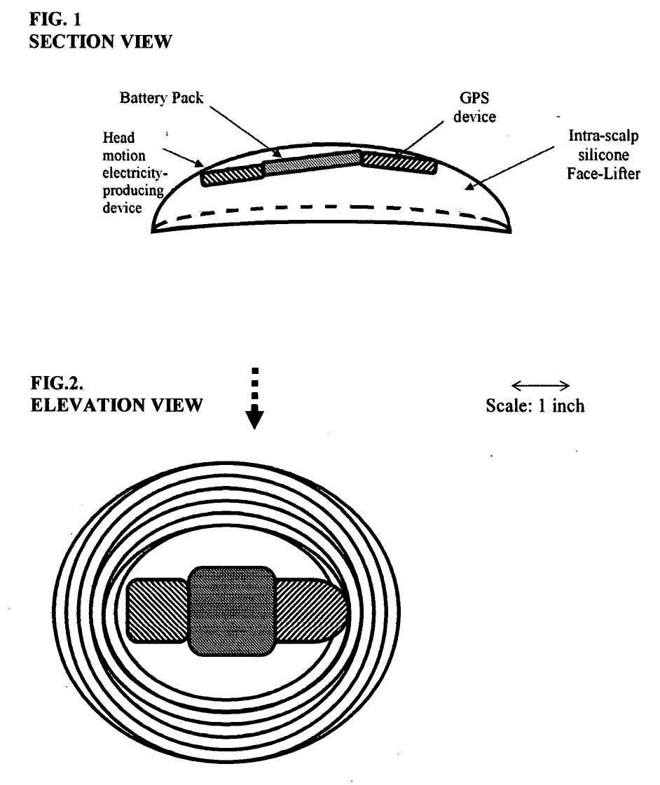

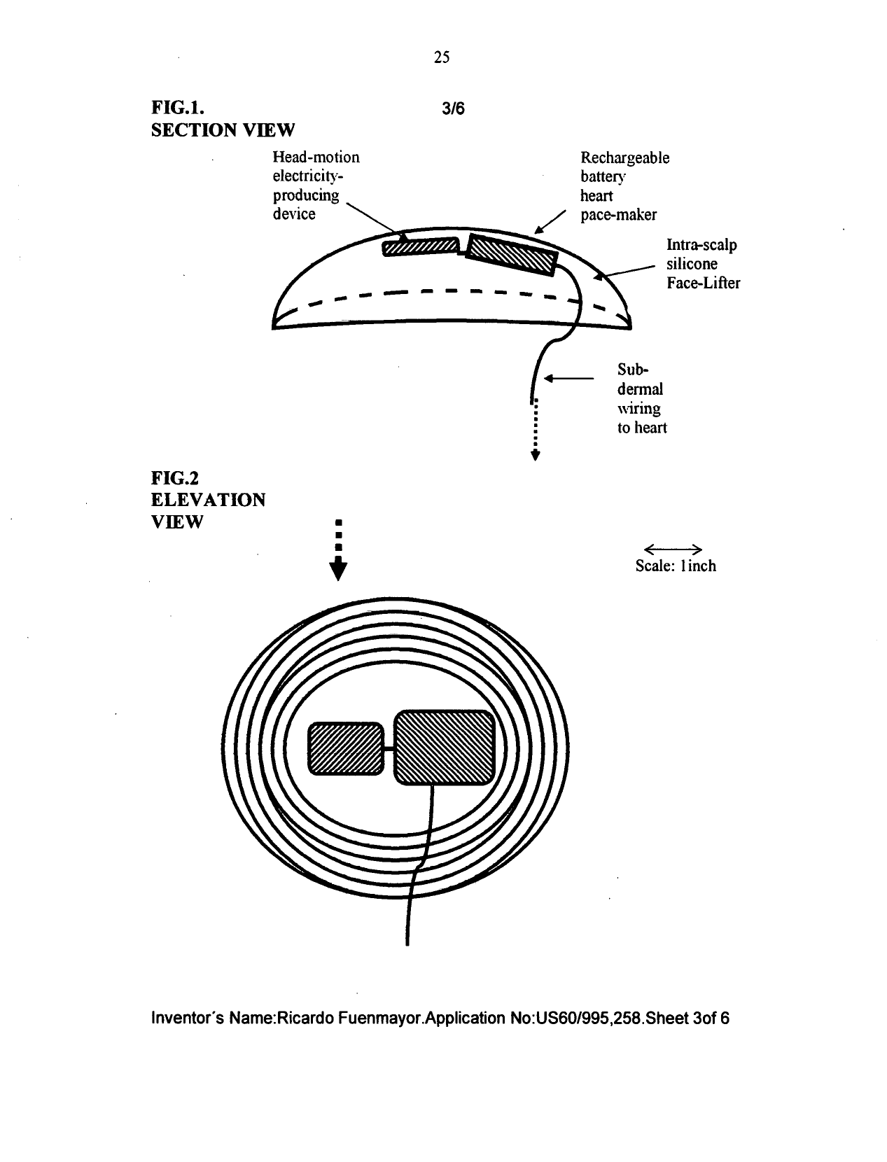

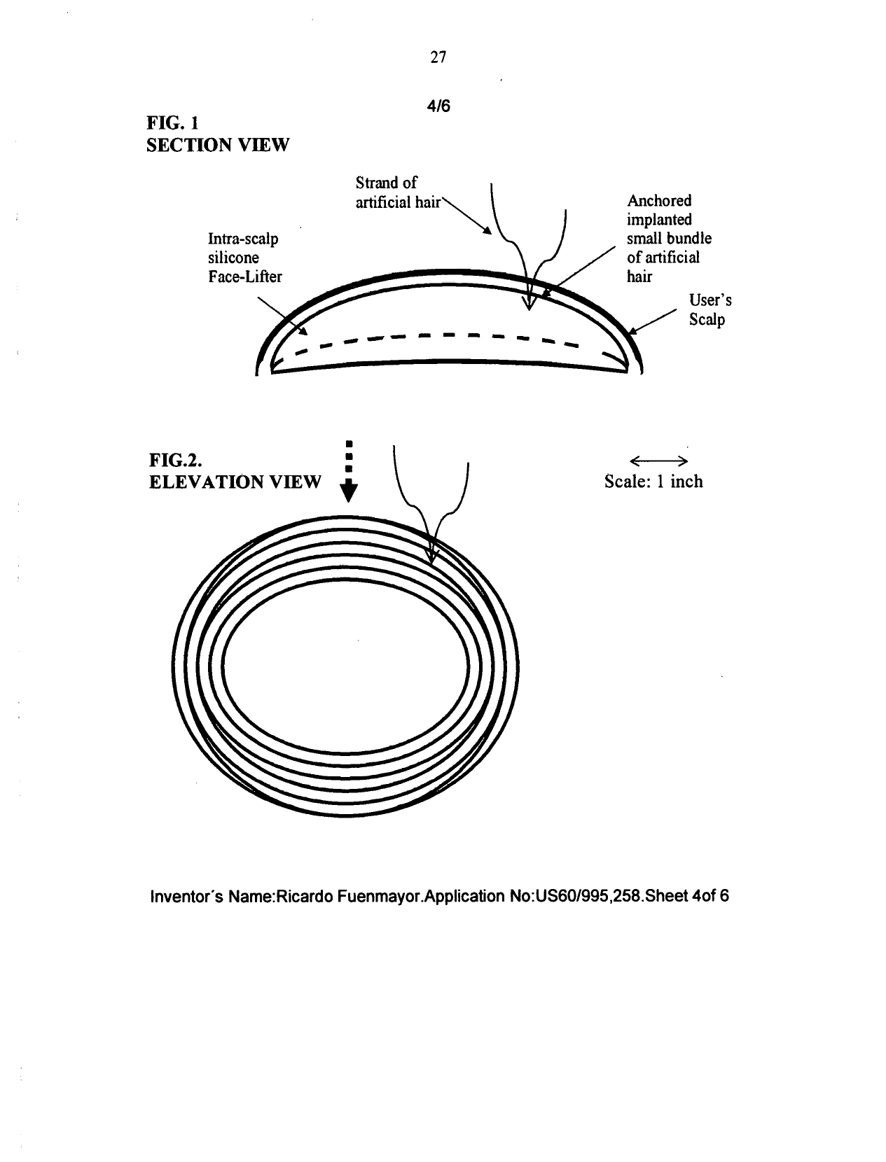

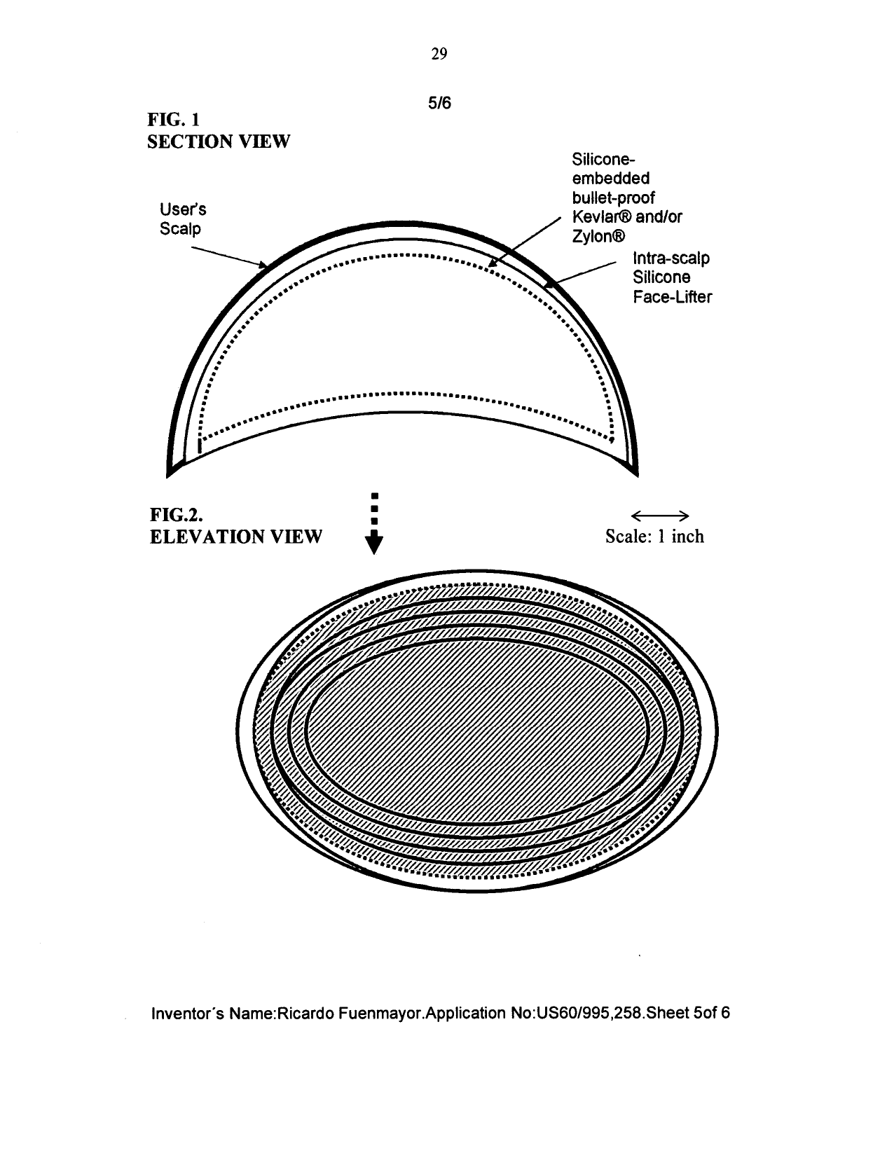

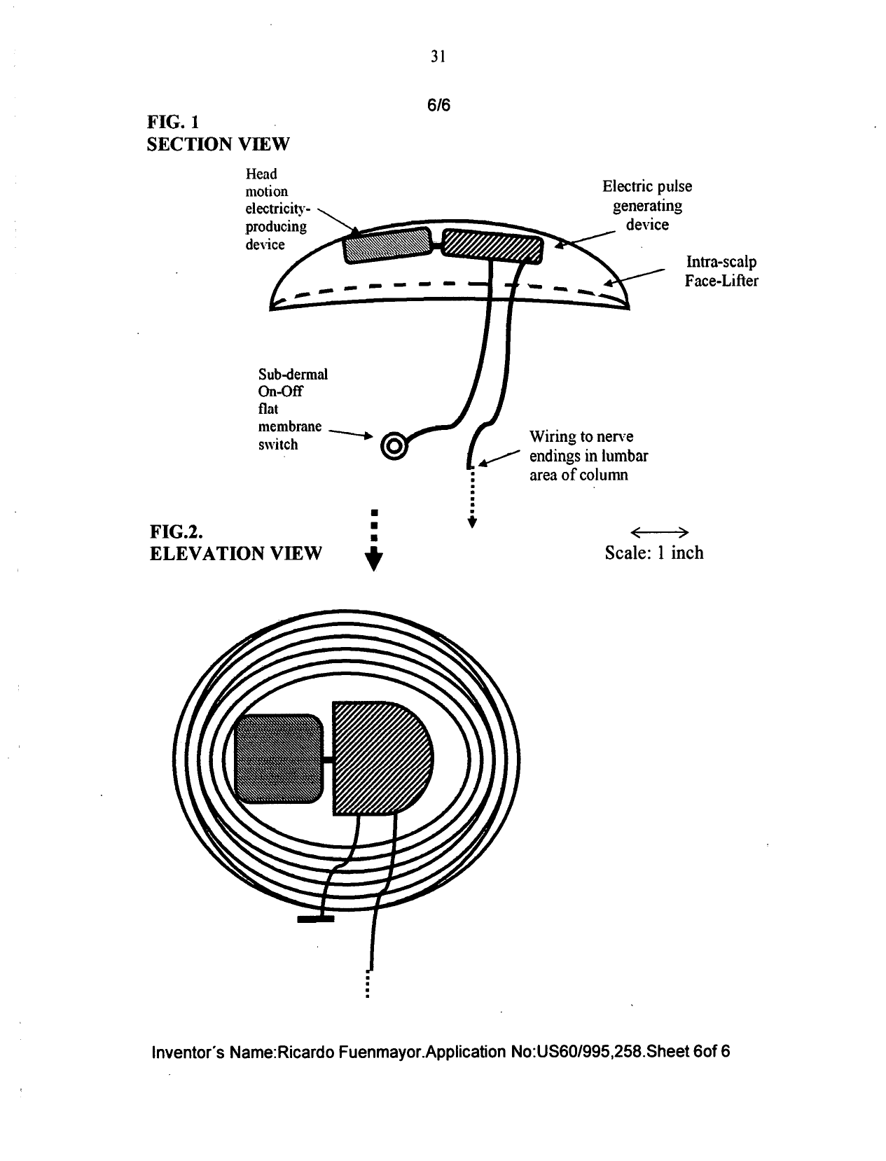

FIG. 1. Section View

It corresponds to a sectional cut through the middle of the device along its longest axis. The improvement consists of a basic intra-scalp Face-Lifter, in whose silicone matrix has been embedded a GPS (Global Positioning System) device and a rechargeable battery (its energy provided by an electricity-producing motion-device similar to Seiko's® “Kinetic”® watches).

FIG. 2. Elevation View

This drawing corresponds to the depiction of the invention as seen from above showing a Face-Lifter used as a stand/support for a GPS device.

Field of Use No. 3: Sheet 3/6. 2 Figures.

FIG. 1. Section View

It corresponds to a sectional cut through the middle of the device along its longest axis. The improvement consists of a basic intra-scalp Face-Lifter, in whose silicone matrix has been embedded a Heart Pace-Maker with rechargeable batteries (its energy provided by an electricity-producing motion-device similar to Seiko's® “Kinetic”® watches) and sub-dermal leading wires that end up in the heart muscle.

FIG. 2. Elevation View

This drawing corresponds to the depiction of the invention as seen from above.

Field of Use No. 4: Sheet 4/6. 2 Figures.

FIG. 1. Section View

It corresponds to a sectional cut through the middle of the device along its longest axis. The improvement consists of using a basic intra-scalp Face-Lifter device as an Artificial Hair Anchoring Base, i.e. using a Face-Lifter as an anchoring base for permanently implanting small bundles of artificial hair.

FIG. 2. Elevation View

This drawing corresponds to the depiction of the invention as seen from above.

Field of Use No. 5: Sheet 5/6. 2 Figures.

FIG. 1. Section View

It corresponds to a sectional cut through the middle of the device along its longest axis. The improvement consists of a thinner version of a silicone Face-Lifter sandwiching a piece of bullet-proof Kevlar® and/or Zylon® fabric, covering, and thus protecting, the temporal, occipital, parietal, and frontal areas of the head

FIG. 2. Elevation View

This drawing corresponds to the depiction of the invention as seen from above.

Field of Use No. 6: Sheet 6/6. 2 Figures.

FIG. 1. Section View

It corresponds to a sectional cut through the middle of the device along its longest axis. The improvement consists of a basic intra-scalp Face-Lifter, in whose silicone matrix has been embedded a small electric pulse-generating device (a small rechargeable battery, its energy provided by a electricity-producing motion-device similar to Seiko's® “Kinetic”® watches), a sub-dermal membrane-type flat switch, and a sub-dermal 20-30 inch-long wiring with a downward descent, reaching the lumbar area of the back for the generation of climaxes in anorgasmic women.

FIG. 2. Elevation View

This drawing corresponds to the depiction of the invention as seen from above.

### DETAILED DESCRIPTION OF THE IMPROVEMENTS OF AN INVENTION

In the next pages there is a detailed description of the six proposed improvements made on an invention—or prior art—entitled “Instant Face-Lifter”. The proposed improvements or different Fields of Use are:

### Field of Use No. 1:

“Intra-scalp TV/Radio/Stereo-Music-Reproducing Device Face-Lifter”:

### Field of Use No. 2:

“Intra-scalp GPS-Face-Lifter”

### Field of Use No. 3:

“Intra-scalp Heart Pace-Maker-Face-Lifter”

### Field of Use No. 4:

“Intra-scalp Artificial Hair Anchoring Base Face-Lifter”.

### Field of Use No. 5:

“Intra-scalp Bullet-Proof Head-Armor Face-Lifter”

### Field of Use No. 6:

“Intra-scalp Orgasm-inducer Face-Lifter”

These six improvements share as common ground the basic structure and design of the Instant Face-Lifter, keeping as main features, its concealment, being totally unnoticed from the outside, and its being a beautifying device to people in their fourth to fifth decade, stretching the skin of the user's face and thus producing a younger look, while adding to its usefulness by serving as a stand or support for other pieces of equipment or different applications. In all six improvements, the invention is slided under the scalp of the user through a 3″ to 4″ long horizontal incision on the parietal area using only local anesthesia. Additionally, for those proposed improvements that work with electric energy and are provided with a rechargeable battery, being the Face-Lifter located on the top of the head, it takes advantage of the continuous rotational head motion of the user to generate energy by means of an ingenious rotating weigh combined with a tiny gearbox that converts few r.p.m. of the weigh's gyration movement into thousands of r.p.m. that impulses a small electricity-generator (a motion-device similar to Seiko's® “Kinetic”® watches) thus obtaining an endless supply of energy.

### Field of Use No. 1

“Intra-scalp TV set/Radio/Stereo-Music-Reproducing Device Face-Lifter”: Using a basic Face-Lifter as a stand or support for an implanted TV signal receptor set and/or an AM-FM Radio Signal Receptor set and/or an Open Platform Stereo Music Producing device (i.e. MP3, MP4, Ipod®, Digital Walkman®, WMA®, etc).

In the Field of Use No. 1, the improvement on the prior art consists of a basic Face-Lifter (“which consists of an intra-scalp oval-shaped device made of solid, medical grade silicone, typically measuring 5 12/16″ long×4½″″ wide, with sloping edges and an ever increasing thickness in the direction from border to center, starting at a negligible thickness at the borders and increasing to a varying maximum thickness—i.e. b 1/2″, 3/4″, 1″, 1 1/2″, etc—at its center as to be able to provide the user the desired amount of skin stretching effect”) to which it has been added an implanted TV signal receptor set and/or an AM-FM Radio Signal Receptor set and/or an Open Platform Stereo Music Producing device (i.e. MP3, MP4, Ipod®, Digital Walkman®, WMA®, etc).

The improvement consists of a basic intra-scalp Face-Lifter, in whose silicone matrix has been embedded: a) a TV signal receiving set, and/or an AM/FM radio signal receptor, and/or an open platform stereo music reproducing device (i.e. MP3, MP4, Apple's® Ipod®, Microsoft's® WMA®, Sony's® Walkman®, etc.) with Bluetooth®-like or similar transmitter; b) a rechargeable battery (its energy provided by an electricity-producing motion-device similar to Seiko's® “Kinetic”® watches), c) two lateral sub-dermal 4″ long×½″ wide silicone side straps, each carrying within an earphone, two on-line flat membrane-type switches (for On-Off function, volume control, silent switch, select mode switch) and wiring that continue their downward descent until reaching both ear canals for stereo listening, and/or d) Bluetooth®-like wireless earpieces (to do away with the use of straps).

Additionally, the improvement presents a handy remote control whereby one can select channels, choose mode of operation, set volume, turn it on-off, etc.

The TV receptor embedded in the face-lifter may transmit TV images either wirelessly (i.e. via Bluetooth® transmitters and earpieces, and/or microwave, infrared or ultrasound waves) or by means of a small wire to a remote few inches wide flat screen mounted on a glasses frame in front of the eyes.

Among its many advantages, this improvement allows the user to listen hand-freely to his/her favorite music in stereo at will, as well as to keep him/her informed at all times, in an unnoticeable fashion, with an all-weather and water-proof technology that may be up-graded with other perks such as “wake-up call” features and others.

### Field of Use No. 2

“Intra-scalp GPS-Face-Lifter”: Using a Face-Lifter as a stand or support for a miniature GPS device.

In the Field of Use No. 2, the improvement on the prior art consists of a basic Face-Lifter (“which consists of an intra-scalp oval-shaped device made of solid, medical grade silicone, typically measuring 5 12/16″ long×4 12/16″ wide, with sloping edges and an ever increasing thickness in the direction from border to center, starting at a negligible thickness at the borders and increasing to a varying maximum thickness—i.e. ½″, 3/4″, 1″, 1 1/2″, etc—at its center as to be able to provide the user the desired amount of skin stretching effect”) used as a stand or support for a miniature GPS device (“Global Positioning System”).

The improvement consists of a basic intra-scalp Face-Lifter, in whose silicone matrix has been embedded a GPS device and a rechargeable battery (its energy provided by an electricity-producing motion-device similar to Seiko's® “Kinetic”® watches).

Advantages: this improvement, completely concealed, allows for a discreet satellital follow-up of all at-risk personnel working or traveling abroad, specially those visiting dangerous places of the world (Iraq, Colombia, etc.). Among its potential users: Diplomatic personnel, Army recruits, war airplane pilots (thus becoming a N.G.L.B. “Nobody Gets Left Behind” device), firemen, armour-car personnel, mine workers, tourists traveling to dangerous places of the world, and mountain climbers. The intra-scalp GPS-Face-Lifter also works well as an anti-kidnapping device, allowing to abort kidnappings by serving as tracking device and allowing for the precise satellital location of missing persons, while keeping its beautifying properties to users in their fourth to fifth decade by gently stretching their face's skin, further reducing eyebrow wrinkles and providing an instant fresher look.

### Field of Use No. 3

“Intra-scalp Heart Pace-Maker Face-Lifter”: using an intra-scalp Face-Lifter as a stand or support for an Implanted Heart Pace-Maker.

In the Field of Use No. 3, the improvement on the prior art consists of the use of a basic Face-Lifter (“which consists of an intra-scalp oval-shaped device made, of sold, medical grade silicone, typically measuring 5 12/16″ long×4 12/16″ wide, with sloping edges and an ever increasing thickness in the direction from border to center, starting at a negligible thickness at the borders and increasing to a varying maximum thickness—i.e. b 1/2″, 3/4″, 1″, 1 1/2″, etc—at its center as to be able to provide the user the desired amount of skin stretching effect”) as a stand or support for an Implanted Heart Pace-Maker.

The improvement consists of a basic intra-scalp Face-Lifter, in whose silicone matrix has been embedded a Heart Pace-Maker with rechargeable batteries (its energy provided by an electricity-producing motion-device similar to Seiko's® “Kinetic”® watches) and sub-dermal leading wires that end up in the heart muscle.

This improvement's novelty consists of its innovative location which brings several important advantages: Because of its concealed location, the pace-maker Face-Lifter goes totally unnoticed, presenting no bulging on the chest area as the conventional model does, thus raising the user's self-esteem. Additionally, the improvement, being located on the top of the head, takes advantage of the continuous rotational head motion to generate energy by means of an ingenious rotating weigh combined with a tiny gearbox that converts few r.p.m. of the weigh's gyration movement into thousands of r.p.m. that impulses a small electricity-generator (a motion-device similar to Seiko's® “Kinetic”® watches). Furthermore, the device, possessing rechargeable batteries, eliminates the need to have periodic surgeries to have the device serviced, and hence removed, at intervals to reinstall fresh batteries.

### Field of Use No. 4

“Intra-scalp Artificial Hair Anchoring Base Face-Lifter”: Using a Face-Lifter as an anchoring base for permanently implanting small bundles of artificial hair.

In the Field of Use No. 4, the improvement on the prior art consists of the use of a basic Face-Lifter (“which consists of an intra-scalp oval-shaped device made of sold, medical grade silicone, typically measuring 5 12/16″ long×4 12/16″ wide, with sloping edges and an ever increasing thickness in the direction from border to center, starting at a negligible thickness at the borders and increasing to a varying maximum thickness—i.e. b 1/2″, 3/4″, 1″, 1 1/2″, etc—at its center as to be able to provide the user the desired amount of skin stretching effect”) as a Anchoring Base for Implanting Small Bundles of Artificial Hair as to allow the user to permanently recover an as abundant amount of hair as he wishes to.

The improvement consists of using a basic intra-scalp Face-Lifter device as an Artificial Hair Anchoring Base, i.e. using the silicone structure of a Face-Lifter as an anchoring base for permanently implanting small bundles of artificial hair. The artificial hair to be implanted comes in small bundles. Each little bundle of artificial hair, containing between 3-12 strands of hair, ends up in a little inverted and sharp plastic hook, very much like a straightened fishing hook. The goal of the inverted sharp hook at the bottom of each bundle is to penetrate through the scalp and into the structure of the Face-Lifter, anchoring itself to its silicon matrix, thus rendering each little bundle as a permanent fixture.

Using this improvement as an Artificial Hair Anchoring Base Face-Lifter benefits the user by stretching the skin of his face, already in his forties, smoothening out expression lines and softening crows feet, while additionally helping raise his self-esteem by rejuvenating him, allowing him to permanently recover a furred or hairy look, as it was in his early youth.

### Field of Use No. 5

“Intra-scalp Bullet-Proof Head-Armor Face-Lifter”: Using a Face-Lifter as a bullet-proof head armor.

Since the skin covering most of the head is loosely bound to the skull bone, our aim is to slip below the scalp a piece of bullet-proof fabric which has been embedded into the structure of a Face-Lifter. In the Field of Use No. 5, the improvement on the prior art consists of using a Face-Lifter as a Bullet-Proof Head Armor Device.

The improvement consists of a thinner version of the under-the-scalp Face-Lifter but covering a greater amount of head surface, as to be able to minimize the possible damages inflicted by a gunshot attack to the head. In its bullet-proof head armor application, a sizeable piece of medical-grade silicone-embedded bullet-proof Kevlar® and/or Zylon® fabric is slid under the scalp in order to surround most of the gray-matter areas of the brain, thus covering, and therefore protecting, the temporal, occipital, parietal, and frontal areas of the head.

Typical users of this improvement would be high-risk Army personnel, as well as Police officers, body guards, key politicians, diplomatic personnel assigned to dangerous places abroad, and anyone who feels that his/her physical integrity is being seriously threatened of suffering bodily harm.

Among its advantages are its concealment, being totally undetected from the outside, its imperviousness, thus serving as a shield as to avoid projectiles from becoming lodged in the skull, and its great capacity to minimize the loss of gray matter.

### Field of Use No. 6

“Intra-scalp Orgasm-inducer Face-Lifter”: using a basic intra-scalp silicone Face-Lifter as a stand for a small-pulse electric generator to be used by anorgasmic women to self-induce climaxes at will when they are doing the sexual act with their husbands.

In the Field of Use No. 6, the improvement on the prior art consists of the use of a basic Face-Lifter (“which consists of an intra-scalp oval-shaped device made of solid, medical grade silicone, typically measuring 5 12/16″ long×4 12/16″ wide, with sloping edges and an ever increasing thickness in the direction from border to center, starting at a negligible thickness at the borders and increasing to a varying maximum thickness—i.e. b 1/2″, 3/4″, 1″, 1 1/2″, etc—at its center as to be able to provide the user the desired amount of skin stretching effect”) as a stand or support for an orgasm-inducing electric device.

The improvement consists of a basic intra-scalp Face-Lifter, in whose silicone matrix has been embedded a small electric pulse-generating device, (being itself, in fact, a small rechargeable battery, its energy provided by a head-motion electricity-producing motion-device similar to Seiko's® “Kinetic”® watches), a sub-dermal membrane-type flat switch placed i.e. near the cheek bone, and a sub-dermal 20-30 inch-long wiring that continues its downward descent until reaching the lumbar area of the back in order for its ending electrodes to be in contact with the appropriate nerves leaving the vertebral column in order to voluntarily induce the stimulation of the vaginal and clitoral nerve endings by means of the activation of a sub-dermal membrane-type flat switch. Every time the sub-dermal switch is activated or pressed, a small mili-ampere current is sent to the nerves that lead to the pleasure centers of the anorgasmic woman's vaginal and clitoral area, thus self-inducing a voluntary orgasm.

The advantages of using a Face-Lifter as an Intra-scalp Orgasm-inducer Face-Lifter are especially obvious to anorgasmic women who, due to a lack of sensitivity or psychological/physiological trauma in that area of their body, become full of apathy toward their husbands, losing an important part of the joy of life due to their lack of an adequate physiological response. Once these women, by simply pressing the sub-dermal membrane-type flat switch on their cheeks at the appropriate moment of the intercourse, receive the benefits of reaching repeated climaxes, their sexual and marital lives will simply change forever for good. They will be able to induce orgasms on themselves by simply pressing a button! Consequently, society at large will benefit, saving from potential breakage a number of marriages due to sexual insatisfaction, and ending up with happier husbands and more fulfilled housewives.

Thus, the advantages of this improvement are its concealment, since the device cannot be noticed from the outside, having no wirings that would give out its presence, therefore raising her self-esteem and confidence; additionally, it gives the women users the ability to control their orgasms, thus becoming self-sufficient and independent of the love-making skill of their sexual partner; and, by using the orgasm-inducing electric device she also gets the benefits of using the Face-Lifter, in whose silicone structure the small-pulse electric generator is embedded; among these advantages: she'd get an instant face-lifting since the device dramatically improves her face, already in her 40's to 50's, by creating a brighter, softer, fresher appearance and restoring a more youthful, rested, and aesthetic look, by elevating drooping eyebrows, softening of forehead frown lines, and smoothing out vertical lines between the eyebrows, with minimum scarring or possibility of deforming hairline.

What is claimed is:

1. Field of Use No. 1 An intra-scalp round or oval-shaped device made of a biocompatible material, typically measuring 5 12/16″ long×4 12/16″ wide, having sloping edges and an ever increasing thickness in the direction from border to center, starting at a negligible thickness at the borders and increasing to a typical maximum thickness of 1 1/2″ at its center, its upper design following a curve which parallels the shape of the skull as for it to be unnoticeable from the outside for providing the user the desired amount of skin stretching effect to pursue an instant face-lifting wherein the improvement comprises of embedding into the intra-scalp face-lifting device's silicone structure means for allowing the reproduction of music or sound and/or video and/or TV images, said embedded means comprising:
a music or sound reproducing device, and/or
an AM/FM radio signal receptor, and/or
a compact TV signal receiving device,
the improvement further comprising:
a rechargeable battery (its energy provided by an electricity-producing motion-device similar to Seiko's® “Kinetic”® watches),
two lateral 4″ long×½″ wide silicone sub-dermal side straps ending up near the ear canal for stereo listening, each strap comprising:
wiring,
an earphone and
two flat membrane-type switches (one for On-Off function and the other for mode selection), and,
a wireless transmitter,
whereby the sound reproducing device may transmit music or sound to the wireless earpieces and the TV signal receiving device to wirelessly transmit TV or video images to a remote few inches wide flat TV screen mounted on a glasses frame in front of the eyes, thus allowing the user to be entertained by hand-freely listening to his/her favorite music at will, as well as to allow him/her to watch TV or videos, in an unnoticeable fashion, with an all-weather and water-proof technology;

2. The device according to claim 1, wherein the embedded music or sound reproducing device is an open platform stereo music reproducing device (i.e. MP3, MP4, Apple's® Ipod®, Microsoft's® WMA®, or Sony's® Digital Walkman®),

3. The device according to claim 1, wherein the wireless transmitter is a Bluetooth® or similar transmitter, whereby the embedded sound reproducing device may wirelessly transmit the music or sound to wireless earpieces, as alternative to the use of the sub-dermal straps, and/or wirelessly transmit TV or video images (via microwave, infrared or ultrasound waves) to a remote few inches wide flat TV screen mounted on a glasses frame in front of the eyes.

4. The device according to claim 1, wherein the improvement can be externally controlled by means of a remote control whereby the user can select channels, choose mode of operation, set volume, and turn on-off the reproducing device.

5. Field of Use No. 2 An intra-scalp round or oval-shaped device made of a solid biocompatible material, typically measuring 5 12/16″ long×4 12/16″ wide, having sloping edges and an ever increasing thickness in the direction from border to center, starting at a negligible thickness at the borders and increasing to a typical maximum thickness of 1½″ at its center, its upper design following a curve which parallels the shape of the skull as for it to be unnoticeable from the outside for providing the user the desired amount of skin stretching effect to pursue an instant face-lifting wherein the improvement comprises of embedding into the face-lifting device's silicone structure means for satellital follow-up of at-risk personnel working on or traveling to dangerous places of the world (i.e. Iraq, Colombia, etc.) and/or as tracking device for the precise satellital location of missing persons, said means comprising:
a GPS (“Global Positioning System”) device and
a rechargeable battery (its energy provided by an electricity-producing motion-device similar to Seiko's® “Kinetic”® watches),
thus allowing for a completely concealed and discreet way to follow-up personnel working on or traveling to dangerous places, being its potential users: Diplomatic and Army Corps personnel, warplane pilots, tourists, firemen, mineworkers and mountain climbers.

6. Field of Use No. 3 An intra-scalp round or oval-shaped device made of a solid biocompatible material, typically measuring 5 12/16″ long×4 12/16″ wide, having sloping edges and an ever increasing thickness in the direction from border to center, starting at a negligible thickness at the borders and increasing to a typical maximum thickness of 1½″ at its center, its upper design following a curve which parallels the shape of the skull as for it to be unnoticeable from the outside for providing the user the desired amount of skin stretching effect to pursue an instant face-lifting wherein the improvement comprises of embedding into the intra-scalp face-lifting device's silicone structure means for administering small and rhythmic electrical pulses to the heart muscle as to keep it pumping blood on a continuous basis, said means comprising:
a Heart Pace-Maker,
a rechargeable battery (its energy provided by an electricity-producing motion-device similar to Seiko's® “Kinetic”® watches), and
sub-dermal leading wires that end up in the heart muscle,
therefore providing the user with a constant and rhythmic heartbeat pattern and, because being the device totally concealed and having a rechargeable battery system, raising the user's self-esteem, as well as eliminating the need to have the device removed at periodic intervals to reinstall fresh batteries, thus eliminating the need for maintenance surgeries.

7. Field of Use No. 4 An intra-scalp round or oval-shaped device made of a solid biocompatible material, typically measuring 5 12/16″ long×4 12/16″ wide, having sloping edges and an ever increasing thickness in the direction from border to center, starting at a negligible thickness at the borders and increasing to a typical maximum thickness of 1½″ at its center, its upper design following a curve which parallels the shape of the skull as for it to be unnoticeable from the outside for providing the user the desired amount of skin stretching effect to pursue an instant face-lifting wherein the improvement comprises of using the intra-scalp silicon matrix of a Face-Lifter device as an Artificial Hair Anchoring Base for permanently implanting small bundles of 3-12 strands of artificial hair provided with inverted sharp plastic hooks at their base, very much like straightened fishing hooks, for piercing them all the way through the scalp and into the silicone structure of the Face-Lifter, in consequence permanently anchoring the small bundles of hair into the silicon matrix, thus raising the user's self-esteem by rejuvenating him, allowing the balding user to recover a furred or hairy look, as it was in his early youth.

8. Field of Use No. 5 An intra-scalp round or oval-shaped device made of a solid biocompatible material, typically measuring 5 12/16″ long×4 12/16″ wide, having sloping edges and an ever increasing thickness in the direction from border to center, starting at a negligible thickness at the borders and increasing to a typical maximum thickness of 1½″ at its center, its upper design following a curve which parallels the shape of the skull as for it to be unnoticeable from the outside for providing the user the desired amount of skin stretching effect to pursue an instant face-lifting wherein the improvement comprises of providing means to protecting the user's head against gunshot attack injuries, said means comprising a thinner and larger version of the Face-Lifter, covering a greater amount of head surface, as to surround most of the gray-matter areas of the brain, and embedding into the intra-scalp face-lifting device's silicone structure a sizeable piece of bullet-proof fabric, thus working as head armor and helping, due to its shielding ability, high-risk Army personnel, Police officers, body guards, key politicians, diplomatic personnel assigned to dangerous places abroad, and anyone who feels that his/her physical integrity is being seriously threatened of suffering bodily harm, diminishing the chances of projectiles entering into the skull bone, and minimizing the loss of gray matter in case of gunshot attack to the head.

9. The device according to claim 8, wherein the bullet-proof fabric consists of a piece of Kevlar® and/or Zylon® fabric, sandwiched within a Face-Lifter, which is implanted under the scalp as to surround most of the gray-matter areas of the brain, thus covering, and therefore protecting, the temporal, occipital, parietal, and frontal areas of the head against gunshot attacks.

10. Field of Use No. 6 An intra-scalp round or oval-shaped device made of a solid biocompatible material, typically measuring 5 12/16″ long×4 12/16″ wide, having sloping edges and an ever increasing thickness in the direction from border to center, starting at a negligible thickness at the borders and increasing to a typical maximum thickness of 1½″ at its center, its upper design following a curve which parallels the shape of the skull as for it to be unnoticeable from the outside for providing the user the desired amount of skin stretching effect to pursue an instant face-lifting wherein the improvement comprises of embedding into the face-lifting device's silicone structure means for administering small intensity electrical pulses for nerve ending stimulation of anorgasmic women's pleasure centers, said means comprising:
a mili-amp electric pulse-generating device, (being itself, in fact, a small rechargeable battery, its energy provided by a electricity-producing motion-device similar to Seiko's® “Kinetic”® watches),
a membrane-type flat switch connected by wiring to the electric pulse-generating device, implanted sub-dermally i.e. near the cheek bone, and,
a sub-dermal 20-30 inch-long wiring,
the latter connecting to the electric pulse-generating device and continuing its downward descent until reaching the appropriate nerves leaving the lumbar area of the vertebral column and leading to the anorgasmic woman's vaginal and clitoral area, who, by pressing the sub-dermal switch, sends a small mili-ampere current to her pleasure centers, as a result, self-inducing a voluntary orgasm, thus raising her self-esteem and confidence, and therefore helping their sexual and marital lives for good, while at the same obtaining the benefits of using the Face-Lifter, whereby she'd get an instant face-lifting, dramatically improving her face, already in her 40's to 50's, by creating a brighter, softer, fresher appearance and restoring a more youthful, rested, and aesthetic look, by elevating drooping eyebrows, softening forehead frown lines, and smoothing out vertical lines between the eyebrows, with minimum scarring or possibility of deforming hairline.
